(19) United States
(12) Patent Application Publication
(10) Pub. No.: US 2013/0254933 A1
(43) Pub. Date: Sep. 26, 2013

(54) CRY1I PROTEINS AND GENES FOR INSECT CONTROL

(71) Applicant: Vance Kramer, Research Triangle Park, NC (US)

(72) Inventor: Vance Kramer, Research Triangle Park, NC (US)

(73) Assignee: SYNGENTA PARTICIPATIONS AG, Research Triangle Park, NC (US)

(21) Appl. No.: 13/989,517

(22) PCT Filed: Nov. 22, 2011
(86) PCT No.: PCT/US2011/061753

§ 371 Date: May 24, 2013

Related U.S. Application Data
(60) Provisional application No. 61/422,328, filed on Dec. 13, 2010.

(51) Int. Cl. C07K 14/325

(52) U.S. Cl. CPC C07K 14/325
USPC 800/279; 530/350; 536/23.71; 435/320.1; 435/419; 435/412; 435/415; 435/414; 800/298; 800/320.1; 800/320.3; 800/320.2; 800/312; 800/317.3; 800/314; 554/1; 127/32; 435/69.1; 800/302; 426/622

(57) ABSTRACT

Novel insecticidal toxins isolated from Bacillus thuringiensis that are active against lepidopteran insect pests are disclosed. The DNA encoding the insecticidal toxins can be used to transform various prokaryotic and eukaryotic organisms to express the insecticidal toxins. These recombinant organisms can be used to control lepidopteran insects in various environments.

### FIELD OF THE INVENTION

This invention relates to the field of molecular biology. Provided are novel genes that encode pesticidal proteins. These proteins and the nucleic acid sequences that encode them are useful in preparing pesticidal formulations and in the production of transgenic pest-resistant plants.

### BACKGROUND

Bacillus thuringiensis (Bt) is a gram-positive spore forming soil bacterium characterized by its ability to produce crystalline inclusions that are specifically toxic to certain orders and species of insects, but are harmless to plants and other non-target organisms. For this reason, compositions including Bacillus thuringiensis strains or their insecticidal proteins can be used as environmentally-acceptable insecticides to control agricultural insect pests or insect vectors for a variety of human or animal diseases.

The use of insecticidal crystal proteins derived from the Bacillus thuringiensis commonly referred to as “Cry proteins” have been utilized. Cry proteins are globular protein molecules which accumulate as protoxins in crystalline form during late stage of the sporulation of Bt. After ingestion by the pest, the crystals are solubilized to release protoxins in the alkaline midgut environment of the larvae. Protoxins (approximately 130-140 kDa) are converted into mature toxic fragments (approximately 60-70 kDa N terminal region) by gut proteases. Many of these proteins are quite toxic to specific target insects, but harmless to plants and other non-targeted organisms.

Cry proteins from Bacillus thuringiensis have potent insecticidal activity against predominantly Lepidopteran, Dipteran, and Coleopteran larvae. These proteins also have shown activity against other insect orders, for example, Hymenoptera, Homoptera, Phthiraptera, Mallophaga, and Acari pest orders, as well as other invertebrate orders such as Nemathelminthes, Platyhelminthes, and Sarcomastigorphora (Feitelson (1993) The Bacillus Thuringiensis family tree. In Advanced Engineered Pesticides. Marcel Dekker, Inc., New York, N.Y.) These proteins were originally classified as CryI to CryVI based primarily on their insecticidal activity. The major classes were Lepidoptera-specific (I), Lepidoptera- and Diptera-specific (II), Coleoptera-specific (III), Diptera-specific (IV), and nematode-specific (V) and (VI). The proteins were further classified into subfamilies; more highly related proteins within each family were assigned divisional letters such as CryIA, CryIB, CryIC, etc. Even more closely related proteins within each division were given names such as CryIC(a), CryIC(b) etc. The terms “Cry toxin” and “delta-endotoxin” are used interchangeably with the term “Cry protein”.

A new nomenclature has been described for the Cry genes based upon amino acid sequence homology rather than insect target specificity (Crickmore et al. (1998) Microbiol. Mol. Biol. Rev. 62:807-813). In this more accepted classification, each toxin is assigned a unique name incorporating a primary rank (an Arabic number), a secondary rank (an uppercase letter), a tertiary rank (a lowercase letter), and a quaternary rank (another Arabic number). In the new classification, Roman numerals have been exchanged for Arabic numerals in the primary rank.

Cry proteins generally have five conserved sequence domains, and three conserved structural domains (see, for example, de Maagd et al. (2001) Trends Genetics 17:193-199). The first conserved structural domain consists of seven alpha helices and is involved in membrane insertion and pore formation. Domain II consists of three beta-sheets arranged in a Greek key configuration, and domain III consists of two antiparallel beta-sheets in ‘jelly-roll’ formation (de Maagd et al., 2001, supra). Domains II and III are involved in receptor recognition and binding, and are therefore considered determinants of toxin specificity.

Other, non-endotoxin genes and the proteins they encode have also been isolated from Bacillus thuringiensis. Unlike the Cry proteins, which are produced during sporulation and are maintained within the cell in a parasporal crystal, these new insecticidal proteins are secreted from Bacillus during the vegetative growth stage and thus have been designated Vegetative Insecticidal Proteins (VIPs). (See for example, U.S. Pat. Nos. 5,877,012; 6,107,279; 6,137,033; 5,849,870 and 5,889,174, incorporated herein by reference).

Cry1Ia is a unique insecticidal protein from Bacillus thuringiensis in that it has biochemical properties similar to both Cry and VIP proteins. Cry1Ia has the conserved domains of other Cry proteins but is not produced in parasporal crystals. Previous reports have suggested the cryptic nature of the cry1Ia-type genes on the basis of the absence of Cry1Ia-type proteins in parasporal crystals. Kostichka et al. (1996. J. Bacteriol. 178:2141-2144) first reported the secretion of Cry1Ia and the presence of an N-terminal domain of a Cry1I that likely acts as a secretion signal peptide. Previous reports have shown that Cry1Ia is active against both lepidopteran and coleopteran insects.

Numerous commercially valuable plants, including common agricultural crops, are susceptible to attack by insect and nematode pests, causing substantial reductions in crop yield and quality. For example, growers of maize (Zea mays), face a major problem with combating pest infestations. Nematodes and insects, including Lepidopteran and Coleopteran insects, annually destroy an estimated 15% of agricultural crops in the United States and an even greater percentage in developing countries. In addition, competition with weeds and parasitic and saprophytic plants account for even more potential yield losses. Yearly, such pests cause over $100 billion in crop damage in the United States alone.

In an effort to combat pest infestations, various methods have been employed in order to reduce or eliminate pests in a particular plot. These efforts include rotating corn with other crops that are not a host for a particular pest and applying pesticides to the above-ground portion of the crop, applying pesticides to the soil in and around the root systems of the affected crop. Traditionally, farmers have relied heavily on chemical pesticides to combat pest damage. However, the use of chemical pesticides is costly, as farmers apply billions of gallons of synthetic pesticides to combat these pests each growing season, costing nearly $8 billion. In addition, such pesticides are inconvenient for farmers, result in the emergence of insecticide-resistant pests, and they raise significant environmental and health concerns.

As a result of these concerns and the costs of pesticides, there is a demand for alternative insecticidal agents for agricultural crops. For example, maize plants incorporating transgenic genes which cause the maize plant to produce insecticidal proteins providing protection against target pest(s) is a more environmentally friendly approach to controlling pests.

Because of the devastation that insects can confer there is a continual need to discover new forms of insecticidal proteins with different modes of action.

### SUMMARY

The invention provides compositions and methods for conferring pest resistance to bacteria, plants, plant cells, tissues and seeds. In particular, novel cry1I nucleic acid sequences isolated from Bacillus thuringiensis, and sequences substantially identical thereto, whose expression results in proteins with toxicity to economically important insect pests, particularly insect pests that infest plants, are provided. The invention is further drawn to the novel Cry1I toxins resulting from the expression of the nucleic acid sequences, and to compositions and formulations containing the Cry1I toxins, which are capable of inhibiting the ability of insect pests to survive, grow and reproduce, or of limiting insect-related damage or loss to crop plants. The invention is also drawn to methods of using the nucleic acid sequences, for example in DNA constructs or expression cassettes for transformation and expression in organisms, including microorganisms and plants. The nucleotide or amino acid sequences may be synthetic sequences that have been designed for expression in an organism including, but not limited to, a microorganism or a plant or in making hybrid toxins with enhanced pesticidal activity. The invention is further drawn to methods of making the toxins and to methods of using the nucleic acid sequences, for example in microorganisms to control insects or in transgenic plants to confer protection from insect damage, and to methods of using the Cry1I toxins, and compositions and formulations comprising the Cry1I toxins, for example applying the Cry1I toxins or compositions or formulations to insect-infested areas, or to prophylactically treat insect-susceptible areas or plants to confer protection against the insect pests. The nucleotide or amino acid sequences may be synthetic sequences that have been designed for expression in an organism including, but not limited to, a microorganism or a plant. The invention also provides transformed bacteria, plants, plant cells, tissues, and seeds comprising the nucleic acid sequences encoding the Cry1I toxins of the invention.

According to one aspect, the invention provides a Cry1I toxin comprising 719 amino acids and having at least 99% identity with SEQ ID NO: 1, wherein the amino acid corresponding to position 140 is glutamic acid (E), the amino acid corresponding to position 184 is threonine (T), the amino acid corresponding to position 233 is aspartic acid (D), the amino acid corresponding to position 329 is isoleucine (I), the amino acid corresponding to position 377 is threonine (T), the amino acid corresponding to position 393 is phenylalanine (F) or leucine (L), the amino acid corresponding to position 549 is leucine (L), and the amino acid corresponding to position 712 is leucine (L) or glutamine (Q).

In another aspect, the invention provides a Cry1I toxin comprising 719 amino acids and having at least 99% identity with SEQ ID NO: 1, wherein the amino acid at position 140 is glutamic acid (E), the amino acid at position 184 is threonine (T), the amino acid at position 233 is aspartic acid (D), the amino acid at position 329 is isoleucine (I), the amino acid at position 377 is threonine (T), the amino acid at position 393 is phenylalanine (F) or leucine (L), the amino acid at position 549 is leucine (L), and the amino acid at position 712 is leucine (L) or glutamine (Q).

In another aspect, the Cry1I toxin is active against insect pests, particularly Lepidopteran insect pests. In another aspect, the Cry1I toxin is active only to Lepidopteran insect pests.

In particular, nucleic acid molecules corresponding to Cry1I sequences are provided. Additionally, amino acid sequences corresponding to the nucleic acid molecules are encompassed. In particular, the invention provides for isolated nucleic acid molecules comprising a nucleotide sequence encoding the amino acid sequence shown in SEQ ID NOs: 1 or 3, or the nucleotide sequence set forth in SEQ ID NOs: 2, 4, 5, or 6, as well as variants and fragments thereof.

Methods are provided for producing the polypeptides of the invention, and for using those polypeptides for controlling or killing a Lepidopteran pest. Methods are provided for using polypeptides of the invention in conjunction with other polypeptides for controlling or killing Lepidopteran and Coleopteran pests.

The compositions and methods of the invention are useful for the production of organisms with pesticide resistance, specifically bacteria and plants. These organisms and compositions derived from them are desirable for agricultural purposes. The compositions of the invention are also useful for generating altered or improved Cry toxins that have pesticidal activity, or for detecting the presence of Cry toxins or nucleic acids in products or organisms.

Another aspect of the invention is a nucleic acid molecule, comprising a nucleotide sequence that encodes a Cry1I toxin of the invention. In another aspect, the nucleotide sequence comprises SEQ ID NO: 2 or SEQ ID NO: 4. In yet another aspect, the nucleotide sequence has been codon optimized for expression in a plant. In still yet another aspect, the nucleotide sequence comprises SEQ ID NO: 5 or SEQ ID NO: 6.

Yet another aspect of the invention is a chimeric gene comprising a heterologous promoter sequence operatively linked to any one of SEQ ID NO: 2, SEQ ID NO: 4, SEQ ID NO: 5, or SEQ ID NO: 6. In yet another aspect, the promoter is a plant-expressible promoter. In still yet another aspect, the promoter is selected from the group consisting of ubiquitin, cmp, corn TrpA, bacteriophage T3 gene 9 5′ UTR, corn sucrose synthetase 1, corn alcohol dehydrogenase 1, corn light harvesting complex, corn heat shock protein, pea small subunit RuBP carboxylase, Ti plasmid mannopine synthase, Ti plasmid nopaline synthase, petunia chalcone isomerase, bean glycine rich protein 1, Potato patatin, lectin, CaMV 35S, and the S-E9 small subunit RuBP carboxylase promoter.

Still yet another aspect of the invention is a recombinant vector comprising the above chimeric gene. In another aspect, the vector is plasmid, cosmid, phagemid, artificial chromosome, phage or viral vector. In another aspect, the vector is comprised in a transgenic non-human host cell. In another aspect, the host cell is a transgenic plant cell. In another aspect, the transgenic plant cell is maize, wheat, rice, soybean, tobacco, or cotton.

Yet another aspect of the invention is a biological sample derived from the above transgenic plant and comprising a Cry1I toxin. In another aspect, the insecticidal protein protects the biological sample from insect infestation. In another aspect, the biological sample is flour, meal, oil, or starch, or a product derived from any of these biological samples.

Still yet another aspect of the invention is a method of providing a farmer with a means of controlling a Lepidopteran insect pest, said method comprising supplying or selling to the farmer plant material, said plant material comprising a nucleic acid molecule capable of expressing the Cry1I toxin, as described above.

Another aspect of the invention is a method of producing a Cry1I toxin of the invention, comprising the steps of: (a) transforming a non-human host cell with a recombinant nucleic acid molecule comprising a nucleotide sequence which codes for the Cry1I toxin; and (b) culturing the host cell of step (a) under conditions in which the host cell expresses the recombinant nucleic acid molecule, thereby producing the Cry1I toxin. In another aspect, the non-human host cell is a plant cell. In another aspect, the plant cell is a maize cell. In another aspect, the recombinant nucleic acid molecule is codon optimized for expression in a plant. In another aspect, the recombinant nucleic acid molecule comprises SEQ ID NO: 2, SEQ ID NO: 4, SEQ ID NO: 5, or SEQ ID NO: 6. In another aspect, the recombinant nucleic acid molecule further comprises a promoter sequence operably linked to said nucleotide sequence to allow expression of the nucleotide sequence and production of the Cry1I toxin by the host cell. In another aspect, the transforming is performed by Agrobacterium-mediated transformation, electroporation, or microprojectile bombardment.

Another aspect of the invention is a method of reducing pest damage in a transgenic plant caused by Lepidopteran insects and Coleopteran insects. This method comprises planting a transgenic plant seed comprising a first transgene and a second transgene, wherein the first transgene causes expression of a Cry1I toxin and wherein the second transgene causes expression of a toxin from Bacillus thuringiensis; thereby reducing damage caused by Lepidopertan insects and Coleopteran insects to a transgenic plant grown from the transgenic plant seed. In another aspect, the Cry1I toxin comprises 719 amino acids and having at least 99% identity with SEQ ID NO: 1, wherein the amino acid corresponding to position 140 is glutamic acid (E), the amino acid corresponding to position 184 is threonine (T), the amino acid corresponding to position 233 is aspartic acid (D), the amino acid corresponding to position 329 is isoleucine (I), the amino acid corresponding to position 377 is threonine (T), the amino acid corresponding to position 393 is phenylalanine (F) or leucine (L), the amino acid corresponding to position 549 is leucine (L), the amino acid corresponding to position 712 is leucine (L) or glutamine (Q). In another aspect, the first transgene comprises a nucleotide sequence selected from the group consisting of SEQ ID NO: 2, SEQ ID NO: 4, SEQ ID NO: 5 and SEQ ID NO: 6. In another aspect, the toxin from Bacillus thuringiensis is a Cry toxin or a VIP toxin. In another aspect, the Cry toxin is a Cry3 toxin. In another aspect, the Cry3 toxin is a modified Cry3A toxin, as described in U.S. Pat. Nos. 7,030,295 and 7,230,167, incorporated herein by reference in their entirety. In another aspect, the transgenic plant is maize, wheat, rice, soybean, tobacco, or cotton.

These and other features, aspects, and advantages of the invention will become better understood with reference to the following detailed description and claims.

### BRIEF DESCRIPTION OF THE SEQUENCES IN THE SEQUENCE LISTING

SEQ ID NO: 1 is the amino acid sequence of a 5618-Cry1Ia toxin.

SEQ ID NO: 2 is a nucleotide sequence encoding SEQ ID NO: 1.

SEQ ID NO: 3 is the amino acid sequence of a 5621-Cry1Ia toxin.

SEQ ID NO: 4 is a nucleotide sequence encoding SEQ ID NO: 3.

SEQ ID NO: 5 is a codon optimized nucleotide sequence encoding a 5618-Cry1Ia toxin.

SEQ ID NO: 6 is a codon optimized nucleotide sequence encoding a 5621-Cry1Ia toxin.

SEQ ID NO: 7 is cry1B coding sequence.

SEQ ID NO: 8 is a cry1Ac promoter

SEQ ID NO: 9 is a primer spanning nucleotides 1-20 of SEQ ID NO: 8

SEQ ID NO: 10 is a primer spanning nucleotides 179-188 of SEQ ID NO: 8

### DEFINITIONS

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulatory DNA sequence will affect the expression level of the coding or structural DNA sequence.

A “chimeric gene” or “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid coding sequence. The regulatory nucleic acid sequence of the chimeric gene is not normally operatively linked to the associated nucleic acid sequence as found in nature.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

As used herein, “codon optimized” means a recombinant, transgenic, or synthetic nucleotide sequence wherein the codons are chosen to reflect the particular codon bias that a host cell may have. This is done in such a way so as to preserve the amino acid sequence of the polypeptide encoded by the codon optimized nucleotide sequence. In certain embodiments, the DNA sequence of the recombinant DNA construct includes sequence that has been codon optimized for the cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, incorporated herein by reference.

To “control” insects means to inhibit, through a toxic effect, the ability of insect pests to survive, grow, feed, and/or reproduce, or to limit insect-related damage or loss in crop plants. To “control” insects may or may not mean killing the insects, although it preferably means killing the insects.

In the context of the invention, “corresponding to” means that when the amino acid sequences of variant Cry1I toxins are aligned with each other, the amino acids that “correspond to” certain enumerated positions in the invention are those that align with these positions in the Cry1I toxin (SEQ ID NO: 1 or SEQ ID NO: 3), but that are not necessarily in these exact numerical positions relative to the particular Cry1I amino acid sequence of the invention.

To “deliver” a toxin means that the toxin comes in contact with an insect, resulting in toxic effect and control of the insect. The toxin can be delivered in many recognized ways, e.g., orally by ingestion by the insect or by contact with the insect via transgenic plant expression, formulated protein composition(s), sprayable protein composition(s), a bait matrix, or any other art-recognized toxin delivery system.

“Effective insect-controlling amount” means that concentration of toxin that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

A “gene” is a defined region that is located within a genome and that, besides the aforementioned coding sequence, may comprise other nucleic acid sequences responsible for the control of the expression, that is to say the transcription and translation, of the coding portion. A gene may also comprise other 5′ and 3′ untranslated sequences and termination sequences. Further elements that may be present are, for example, introns.

“Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

A “gut protease” is a protease naturally found in the digestive tract of an insect. This protease is usually involved in the digestion of ingested proteins.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

“Homologous recombination” is the reciprocal exchange of nucleic acid fragments between homologous nucleic acid molecules.

“Insecticidal” is defined as a toxic biological activity capable of controlling insects, preferably by killing them.

An “isolated” nucleic acid molecule or an isolated toxin is a nucleic acid molecule or toxin that, by the hand of man, exists apart from its native environment and is therefore not a product of nature. An isolated nucleic acid molecule or toxin may exist in a purified form or may exist in a non-native environment such as a recombinant host cell or a transgenic plant.

A “nucleic acid molecule” or “nucleic acid sequence” is single- or double-stranded DNA or RNA that can be isolated from any source. In the context of the present invention, the nucleic acid molecule is preferably a segment of DNA.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A “promoter” is an untranslated DNA sequence upstream of the coding region that contains the binding site for RNA polymerase and initiates transcription of the DNA. The promoter region may also include other elements that act as regulators of gene expression.

A “protoplast” is an isolated plant cell without a cell wall or with only parts of the cell wall.

“Regulatory elements” refer to sequences involved in controlling the expression of a nucleotide sequence. Regulatory elements comprise a promoter operably linked to the nucleotide sequence of interest and termination signals. They also typically encompass sequences required for proper translation of the nucleotide sequence.

Substantially identical: the phrase “substantially identical,” in the context of two nucleic acid or protein sequences, refers to two or more sequences or subsequences that have at least 60%, preferably 80%, more preferably 90, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (National Center for Biotechnology Information, U.S. National Library of Medicine, 8600 Rockville Pike, Bethesda, Md. 20894 USA). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but not to other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

Nucleotides are indicated by their bases by the following standard abbreviations: adenine (A), cytosine (C), thymine (T), and guanine (G). Amino acids are likewise indicated by the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (H is; H), isoleucine (Ile; l), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

### DETAILED DESCRIPTION OF THE INVENTION

This invention relates to a Cry1I toxin, nucleotide sequences which encode the toxin, and to the making and using of the Cry1I toxin to control Lepidopteran insect pests.

According to one embodiment, the invention provides a Cry1I toxin comprising 719 amino acids and having at least 99% identity with SEQ ID NO: 1, wherein the amino acid corresponding to position 140 is glutamic acid (E), the amino acid corresponding to position 184 is threonine (T), the amino acid corresponding to position 233 is aspartic acid (D), the amino acid corresponding to position 329 is isoleucine (I), the amino acid corresponding to position 377 is threonine (T), the amino acid corresponding to position 393 is phenylalanine (F) or leucine (L), the amino acid corresponding to position 549 is leucine (L), and the amino acid corresponding to position 712 is leucine (L) or glutamine (Q).

Another embodiment of the invention is a nucleic acid molecule, comprising a nucleotide sequence that encodes a Cry1I toxin 719 amino acids and having at least 99% identity with SEQ ID NO: 1, wherein the amino acid at position 140 is glutamic acid (E), the amino acid at position 184 is threonine (T), the amino acid at position 233 is aspartic acid (D), the amino acid at position 329 is isoleucine (I), the amino acid at position 377 is threonine (T), the amino acid at position 393 is phenylalanine (F) or leucine (L), the amino acid at position 549 is leucine (L), the amino acid at position 712 is leucine (L) or glutamine (Q). In another embodiment, the nucleotide sequence comprises a sequence selected from the group consisting of SEQ ID NO: 2 and SEQ ID NO: 4. In yet another embodiment, the nucleotide sequence is codon optimized for expression in a plant. In still yet another embodiment, the codon optimized sequence comprises a sequence selected from the group consisting of SEQ ID NO: 5 and SEQ ID NO: 6. In another embodiment, the Cry1I toxin is active against Lepidopteran insects and is not active against Coleopteran insects.

Yet another embodiment of the invention is a chimeric gene comprising a heterologous promoter sequence operatively linked to a nucleic acid sequence encoding said Cry1I toxin, or any one of SEQ ID NO: 2, SEQ ID NO: 4, SEQ ID NO: 5, or SEQ ID NO: 6. In yet another embodiment, said promoter is a plant-expressible promoter. In still yet another embodiment, the plant-expressible promoter is selected from the group consisting of ubiquitin, cmp, corn TrpA, bacteriophage T3 gene 9 5′ UTR, corn sucrose synthetase 1, corn alcohol dehydrogenase 1, corn light harvesting complex, corn heat shock protein, pea small subunit RuBP carboxylase, Ti plasmid mannopine synthase, Ti plasmid nopaline synthase, petunia chalcone isomerase, bean glycine rich protein 1, Potato patatin, lectin, CaMV 35S, and the S-E9 small subunit RuBP carboxylase promoter.

Still yet another embodiment of the invention is a recombinant vector comprising a nucleic acid sequence encoding said Cry1I toxin, or any one of SEQ ID NO: 2, SEQ ID NO: 4, SEQ ID NO: 5, or SEQ ID NO: 6. In another embodiment, the vector is further defined as a plasmid, cosmid, phagemid, artificial chromosome, phage or viral vector. In yet another embodiment, the vector is comprised in a transgenic non-human host cell. In still yet another embodiment, the host cell is a transgenic plant cell. In further yet another embodiment, the transgenic plant cell is maize, wheat, rice, soybean, tobacco, or cotton.

In another embodiment of the invention, a Cry1I toxin of the invention is expressed in a higher organism, e.g., a plant. In this case, transgenic plants expressing effective amounts of the toxin protect themselves from insect pests. When the insect starts feeding on such a transgenic plant, it also ingests the expressed toxin. This will deter the insect from further biting into the plant tissue or may even harm or kill the insect. A nucleotide sequence of the invention is inserted into an expression cassette, which is then stably integrated in the genome of the plant. In another embodiment, the nucleotide sequence is included in a non-pathogenic self-replicating virus. Plants transformed in accordance with the invention may be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees.

Once a desired nucleotide sequence has been transformed into a particular plant species, it may be propagated in that species or moved into other varieties of the same species, particularly including commercial varieties, using traditional breeding techniques.

A nucleotide sequence of the invention is expressed in transgenic plants, thus causing the biosynthesis of the corresponding Cry1I toxin in the transgenic plants. In this way, transgenic plants with enhanced resistance to insects are generated. For their expression in transgenic plants, the nucleotide sequences of the invention may require modification and optimization. Although in many cases genes from microbial organisms can be expressed in plants at high levels without modification, low expression in transgenic plants may result from microbial nucleotide sequences having codons that are not preferred in plants. It is known in the art that all organisms have specific preferences for codon usage, and the codons of the nucleotide sequences described in this invention can be changed to conform with plant preferences, while maintaining the amino acids encoded thereby. Furthermore, high expression in plants is best achieved from coding sequences that have at least about 35% GC content, or at least about 45%, or at least about 50%, or at least about 60%. Microbial nucleotide sequences that have low GC contents may express poorly in plants due to the existence of ATTTA motifs that may destabilize messages, and AATAAA motifs that may cause inappropriate polyadenylation. Although certain gene sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). In addition, the nucleotide sequences are screened for the existence of illegitimate splice sites that may cause message truncation. All changes required to be made within the nucleotide sequences such as those described above are made using well known techniques of site directed mutagenesis, PCR, and synthetic gene construction using the methods described in the published patent applications EP 0 385 962 (to Monsanto), EP 0 359 472 (to Lubrizol), and WO 93/07278 (to Ciba-Geigy).

In one embodiment of the invention synthetic genes are made according to the procedure disclosed in U.S. Pat. No. 5,625,136, herein incorporated by reference. In this procedure, maize preferred codons, i.e., the single codon that most frequently encodes that amino acid in maize, are used. The maize preferred codon for a particular amino acid can be derived, for example, from known gene sequences from maize. Maize codon usage for 28 genes from maize plants is found in Murray et al., Nucleic Acids Research 17:477-498 (1989), the disclosure of which is incorporated herein by reference. Specifically exemplified synthetic sequences of the present invention made with maize optimized codons are set forth in SEQ ID NOs: 5 and 6.

In this manner, the nucleotide sequences can be optimized for expression in any plant. It is recognized that all or any part of the gene sequence may be optimized or synthetic. That is, synthetic or partially optimized sequences may also be used.

For efficient initiation of translation, sequences adjacent to the initiating methionine may require modification. For example, they can be modified by the inclusion of sequences known to be effective in plants. Joshi has suggested an appropriate consensus for plants (NAR 15:6643-6653 (1987)) and Clonetech suggests a further consensus translation initiator (1993/1994 catalog, page 210). These consensuses are suitable for use with the nucleotide sequences of this invention. The sequences are incorporated into constructions comprising the nucleotide sequences, up to and including the ATG (while leaving the second amino acid unmodified), or alternatively up to and including the GTC subsequent to the ATG (with the possibility of modifying the second amino acid of the transgene).

The novel cry1I toxin coding sequences of the invention, either as their native sequence or as optimized synthetic sequences as described above, can be operably fused to a variety of promoters for expression in plants including constitutive, inducible, temporally regulated, developmentally regulated, chemically regulated, tissue-preferred and tissue-specific promoters to prepare recombinant DNA molecules, i.e., chimeric genes. The choice of promoter will vary depending on the temporal and spatial requirements for expression, and also depending on the target species. Thus, expression of the nucleotide sequences of this invention in leaves, in stalks or stems, in ears, in inflorescences (e.g. spikes, panicles, cobs, etc.), in roots, and/or seedlings is preferred. In many cases, however, protection against more than one type of insect pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

Examples of constitutive promoters useful in the invention include the CaMV 35S and 19S promoters (Fraley et al., U.S. Pat. No. 5,352,605, incorporated herein by reference). Additionally, a promoter is derived from any one of several of the actin genes, which are expressed in most cell types. The promoter expression cassettes described by McElroy et al. (Mol. Gen. Genet. 231: 150-160 (1991)) can be easily modified for the expression of the novel toxin gene and are particularly suitable for use in monocotyledonous hosts. Yet another constitutive promoter is derived from ubiquitin, which is another gene product known to accumulate in many cell types. A ubiquitin promoter has been cloned from several species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter has been developed in transgenic monocot systems and its sequence and vectors constructed for monocot transformation are disclosed in the patent publication EP 0 342 926. The ubiquitin promoter is suitable for the expression of the novel toxin gene in transgenic plants, especially monocotyledons.

Tissue-specific or tissue-preferential promoters useful for the expression of the novel cry1I toxin coding sequences of the invention in plants, particularly maize, are those that direct expression in root, pith, leaf or pollen. Such promoters are disclosed in WO 93/07278, herein incorporated by reference in its entirety. Other tissue specific promoters useful in the present invention include the cotton rubisco promoter disclosed in U.S. Pat. No. 6,040,504; the rice sucrose synthase promoter disclosed in U.S. Pat. No. 5,604,121; and the cestrum yellow leaf curling virus promoter disclosed in WO 01/73087, all incorporated by reference. Chemically inducible promoters useful for directing the expression of the novel toxin gene in plants are disclosed in U.S. Pat. No. 5,614,395 herein incorporated by reference in its entirety.

The nucleotide sequences of this invention can also be expressed under the regulation of promoters that are chemically regulated. This enables the Cry1I toxins to be synthesized only when the crop plants are treated with the inducing chemicals. Preferred technology for chemical induction of gene expression is detailed in the published application EP 0 332 104 (to Ciba-Geigy) and U.S. Pat. No. 5,614,395. A preferred promoter for chemical induction is the tobacco PR-1a promoter.

Another category of promoters useful in the invention is that which is wound inducible. Numerous promoters have been described which are expressed at wound sites and also at the sites of phytopathogen infection. Ideally, such a promoter should only be active locally at the sites of infection, and in this way the insecticidal toxins only accumulate in cells that need to synthesize the insecticidal toxins to kill the invading insect pest. Preferred promoters of this kind include those described by Stanford et al. Mol. Gen. Genet. 215:200-208 (1989), Xu et al. Plant Molec. Biol. 22:573-588 (1993), Logemann et al. Plant Cell 1:151-158 (1989), Rohrmeier & Lehle, Plant Molec. Biol. 22:783-792 (1993), Firek et al. Plant Molec. Biol. 22:129-142 (1993), and Warner et al. Plant J. 3:191-201 (1993).

Promoters that cause tissue specific expression patterns that are useful in the invention include green tissue specific, root specific, stem specific, and flower specific. Promoters suitable for expression in green tissue include many that regulate genes involved in photosynthesis and many of these have been cloned from both monocotyledons and dicotyledons. One such promoter is the maize PEPC promoter from the phosphoenol carboxylase gene (Hudspeth & Grula, Plant Molec. Biol. 12:579-589 (1989)). Another promoter for root specific expression is that described by de Framond (FEBS 290:103-106 (1991); EP 0 452 269). A preferred stem specific promoter is that described in U.S. Pat. No. 5,625,136 and which drives expression of the maize trpA gene.

Further embodiments of the invention are transgenic plants expressing the nucleotide sequences in a wound-inducible or pathogen infection-inducible manner.

In addition to the selection of a suitable promoter, constructions for expression of an insecticidal toxin in plants require an appropriate transcription terminator to be attached downstream of the heterologous nucleotide sequence. Several such terminators are available and known in the art (e.g. tml from CaMV, E9 from rbcS). Any available terminator known to function in plants can be used in the context of this invention.

Numerous other sequences can be incorporated into expression cassettes described in this invention. These include sequences that have been shown to enhance expression such as intron sequences (e.g. from Adhl and bronzel) and viral leader sequences (e.g. from TMV, MCMV and AMV).

It may be preferable to target expression of the nucleotide sequences of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, the DNA encoding the target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention is also targeted to the endoplasmic reticulum or to the vacuoles of the host cells. Techniques to achieve this are well known in the art.

Numerous transformation vectors available for plant transformation are known to those of ordinary skill in the plant transformation art, and the nucleic acid molecules of the invention can be used in conjunction with any such vectors. The selection of vector will depend upon the preferred transformation technique and the target plant species for transformation. For certain target species, different antibiotic or herbicide selection markers may be preferred. Selection markers used routinely in transformation include the nptII gene, which confers resistance to kanamycin and related antibiotics (Messing & Vierra., 1982. Gene 19: 259-268; and Bevan et al., 1983. Nature 304:184-187), the bar gene, which confers resistance to the herbicide phosphinothricin (White et al., 1990. Nucl. Acids Res 18: 1062, and Spencer et al., 1990. Theor. Appl. Genet. 79: 625-631), the hph gene, which confers resistance to the antibiotic hygromycin (Blochinger & Diggelmann, Mol Cell Biol 4: 2929-2931), and the dhfr gene, which confers resistance to methatrexate (Bourouis et al., 1983. EMBO J. 2(7): 1099-1104), the EPSPS gene, which confers resistance to glyphosate (U.S. Pat. Nos. 4,940,935, and 5,188,642), and the mannose-6-phosphate isomerase gene, which provides the ability to metabolize mannose (U.S. Pat. Nos. 5,767,378 and 5,994,629). The choice of selectable marker is not, however, critical to the invention.

In another embodiment, a nucleotide sequence of the invention is directly transformed into the plastid genome. A major advantage of plastid transformation is that plastids are generally capable of expressing bacterial genes without substantial modification, and plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Natl. Acad. Sci. USA 91, 7301-7305. The basic technique for chloroplast transformation involves introducing 1 to 1.5 kb regions of cloned plastid DNA, termed targeting sequences, flanking a selectable marker together with the gene of interest into a suitable target tissue, e.g., using biolistics or protoplast transformation (e.g., calcium chloride or PEG mediated transformation). The targeting sequences facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifing enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

Yet another embodiment of the invention is a biological sample derived from the above transgenic plant and comprising the Cry1I toxin. In yet another embodiment, the insecticidal protein protects the biological sample from insect infestation. In still yet another embodiment, the biological sample is selected from the group consisting of flour, meal, oil, and starch, or a product derived therefrom.

Another embodiment of the invention is a method of providing a farmer with a means of controlling a Lepidopteran insect pest, the method comprising supplying or selling to the farmer plant material, the plant material comprising a nucleic acid molecule capable of expressing the Cry1I toxin, as described above.

Another embodiment of the invention is a method of producing the Cry1I toxin of the invention, comprising the steps of: (a) transforming a non-human host cell with a recombinant nucleic acid molecule comprising a nucleotide sequence which codes for the Cry1I toxin; and (b) culturing the host cell of step (a) under conditions in which the host cell expresses the recombinant nucleic acid molecule, thereby producing the Cry1I toxin. In another embodiment, the non-human host cell is a plant cell. In another embodiment, the plant cell is a maize cell. In another embodiment, the recombinant nucleic acid molecule is codon optimized for expression in a plant. In another embodiment, the recombinant nucleic acid molecule comprises a sequence selected from the group consisting of SEQ ID NO: 2, SEQ ID NO: 4, SEQ ID NO: 5, and SEQ ID NO: 6. In another embodiment, the recombinant nucleic acid molecule further comprises a promoter sequence operably linked to said nucleotide sequence to allow expression of the nucleotide sequence and production of the Cry1I toxin by the host cell. In another embodiment, the transforming is performed by Agrobacterium-mediated transformation, electroporation, or microprojectile bombardment.

Another embodiment of the invention is a method of reducing pest damage in a transgenic plant caused by Lepidopteran insects and Coleopteran insects, the method comprising planting a transgenic plant seed comprising a first transgene and a second transgene, wherein the first transgene causes expression of a Cry1I toxin and wherein the second transgene causes expression of a toxin from Bacillus thuringiensis; thereby reducing damage caused by Lepidopertan insects and Coleopteran insects to a transgenic plant grown from the transgenic plant seed. In another embodiment, the Cry1I toxin comprises 719 amino acids and having at least 99% identity with SEQ ID NO: 1, wherein the amino acid corresponding to position 140 is glutamic acid (E), the amino acid corresponding to position 184 is threonine (T), the amino acid corresponding to position 233 is aspartic acid (D), the amino acid corresponding to position 329 is isoleucine (I), the amino acid corresponding to position 377 is threonine (T), the amino acid corresponding to position 393 is phenylalanine (F) or leucine (L), the amino acid corresponding to position 549 is leucine (L), the amino acid corresponding to position 712 is leucine (L) or glutamine (Q). In another embodiment, the first transgene comprises a nucleotide sequence selected from the group consisting of SEQ ID NO: 2, SEQ ID NO: 4, SEQ ID NO: 5 and SEQ ID NO: 6. In another embodiment, the toxin from Bacillus thuringiensis is a Cry toxin or a VIP toxin. In another embodiment, the Cry toxin is a Cry3 toxin. In another embodiment, the Cry3 toxin is a modified Cry3A toxin. In another embodiment, the transgenic plant is maize, wheat, rice, soybean, tobacco, or cotton. In another embodiment, the transgenic plant is maize.

The Cry1I toxins of the invention can be used in combination with Bacillus thuringiensis Cry toxins or other pesticidal principles to increase pest target range. Furthermore, the use of the Cry1I toxins of the invention in combination with Bt Cry toxins or other pesticidal principles of a distinct nature has particular utility for the prevention and/or management of insect resistance. Other insecticidal principles include protease inhibitors (both serine and cysteine types), lectins, alpha-amylase, peroxidase and cholesterol oxidase. Vegetative Insecticidal Proteins, such as Vip1Aa and Vip2Aa or Vip3 are also useful in the present invention.

This co-expression of more than one insecticidal principle in the same transgenic plant can be achieved by genetically engineering a plant to contain and express all the genes necessary. Alternatively, a plant, Parent 1, can be genetically engineered for the expression of genes of the present invention. A second plant, Parent 2, can be genetically engineered for the expression of a supplemental insect control principle. By crossing Parent 1 with Parent 2, progeny plants are obtained which express all the genes introduced into Parents 1 and 2.

Transgenic plants or transgenic seed of the invention can also be treated with a pesticidal active ingredient. Where both the pesticidal active ingredient and the transgenic plant or transgenic seed of the invention are active against the same target insect, the combination is useful (i) in a method for enhancing activity of an Cry1I toxin of the invention against the target insect and (ii) in a method for preventing development of resistance to an Cry1I toxin of the invention by providing a second mechanism of action against the target insect. Thus, the invention provides a method of enhancing activity against or preventing development of resistance in a target insect, for example corn rootworm, comprising applying an insecticidal seed coating as described in U.S. Pat. Nos. 5,849,320 and 5,876,739, herein incorporated by reference to a transgenic seed comprising one or more Cry1I toxin of the invention.

Even where the pesticidal active ingredient is active against a different insect or other pest, the pesticidal active ingredient is useful to expand the range of pest control. For example by applying a pesticidal active ingredient that has activity against lepidopteran insects, to mites, to nematodes, and the like to a transgenic plant or seed of the invention, which has activity against coleopteran insects, the transgenic plant or coated transgenic seed produced controls a broader spectrum of crop pest than the transgenic plant or seed alone. Therefore in one embodiment, the invention encompasses a method of controlling crop pests by providing a transgenic plant or transgenic seed of the invention and applying to the transgenic plant or seed an active ingredient.

Such active ingredients that can be applied to a transgenic plant and/or a transgenic seed of the invention as described above includes, without limitation, (1) Acetylcholine esterase (AChE) inhibitors, for example carbamates, for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC and xylylcarb; or organophosphates, for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-5-methyl, demeton-5-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, trichlorfon, vamidothion, and imicyafos. (2) GABA-gated chloride channel antagonists, for example organochlorines, for example camphechlor, chlordane, endosulfan, gamma-HCH, HCH, heptachlor, lindane and methoxychlor; or fiproles (phenylpyrazoles), for example acetoprole, ethiprole, fipronil, pyrafluprole, pyriprole, vaniliprole. (3) Sodium-channel modulators/voltage-dependent sodium channel blockers, for example pyrethroids, for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrin (pyrethrum), eflusilanat; DDT; or methoxychlor. (4) Nicotinergic acetylcholine receptor agonists/antagonists, for example Chloronicotinyls, for example acetamiprid, clothianidin, dinotefuran, imidacloprid, imidaclothiz, nitenpyram, nithiazine, thiamethoxam, AKD-1022, nicotine, bensultap, cartap, thiosultap-sodium, and thiocylam. (5) Allosteric acetylcholine receptor modulators (agonists), for example spinosyns, for example spinosad and spinetoram. (6) Chloride channel activators, for example mectins/macrolides, for example abamectin, emamectin, emamectin benzoate, ivermectin, lepimectin, and milbemectin; or juvenile hormone analogues, for example hydroprene, kinoprene, methoprene, epofenonane, triprene, fenoxycarb, pyriproxifen, and diofenolan. (7) Active ingredients with unknown or nonspecific mechanisms of action, for example fumigants, for example methyl bromide, chloropicrin and sulphuryl fluoride; selective antifeedants, for example cryolite, pymetrozine, pyrifluquinazon and flonicamid; or mite growth inhibitors, for example clofentezine, hexythiazox, etoxazole. (8) Inhibitors of oxidative phosphorylation, ATP disruptors, for example diafenthiuron; organotin compounds, for example azocyclotin, cyhexatin and fenbutatin oxide; or propargite, tetradifon. (9) Oxidative phosphorylation decouplers which interrupt the H-proton gradient, for example chlorfenapyr, binapacyrl, dinobuton, dinocap and DNOC. (10) Microbial disruptors of the insect gut membrane, for example Bacillus thuringiensis strains. (11) Chitin biosynthesis inhibitors, for example benzoylureas, for example bistrifluoron, chlorfluazuron, diflubenzuron, fluazuron, flucycloxuron, flufenoxuron, hexaflumuron, lufenuron, novaluron, noviflumuron, penfluoron, teflubenzuron or triflumuron. (12) Buprofezin. (13) Moulting disruptors, for example cyromazine. (14) Ecdysone agonists/disruptors, for example diacylhydrazines, for example chromafenozide, halofenozide, methoxyfenozide, tebufenozide, and fufenozide (JS118); or azadirachtin. (15) Octopaminergic agonists, for example amitraz; (16) Site III electron transport inhibitors/site II electron transport inhibitors, for example hydramethylnon; acequinocyl; fluacrypyrim; or cyflumetofen and cyenopyrafen. (17) Electron transport inhibitors, for example site I electron transport inhibitors from the group of the METI acaricides, for example fenazaquin, fenpyroximate, pyrimidifen, pyridaben, tebufenpyrad, tolfenpyrad, and rotenone; or voltage-dependent sodium channel blockers, for example indoxacarb and metaflumizone. (18) Fatty acid biosynthesis inhibitors, for example tetronic acid derivatives, for example spirodiclofen and spiromesifen; or tetramic acid derivatives, for example spirotetramat. (19) Neuronal inhibitors with unknown mechanism of action, for example bifenazate. (20) Ryanodin receptor effectors, for example diamides, for example flubendiamide, (R)-, (S)-3-chloro-N.sup.1-{2-methyl-4-[1,2,2,2-tetrafluoro-1-(trifluoromethyl)-ethyl]phenyl}-N.sup.2-(1-methyl-2-methylsulphonylethyl)phthalamide, chlorantraniliprole (Rynaxypyr), or cyantraniliprole (Cyazypyr). (21) Further active ingredients with unknown mechanism of action, for example amidoflumet, benclothiaz, benzoximate, bromopropylate, buprofezin, chinomethionat, chlordimeform, chlorobenzilate, clothiazoben, cycloprene, dicofol, dicyclanil, fenoxacrim, fentrifanil, flubenzimine, flufenerim, flutenzin, gossyplure, japonilure, metoxadiazone, petroleum, potassium oleate, pyridalyl, sulfluramid, tetrasul, triarathene, or verbutin; or the following known active compounds: 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), 4-{[(6-chloropyrid-3-yl)methyl](2,2-difluoroethyl)amino}furan-2(5H)-one (known from WO 2007/115644), 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-on-e (known from WO 2007/115643), 4-{[(6-chloropyrid-3-yl)methyl](cyclopropyl)amino}furan-2(5H)-one (known from EP-A-0 539 588), 4-{[(6-chloropyrid-3-yl)methyl](methyl)amino}furan-2(5H)-one (known from EP-A-0 539 588), [(6-chloropyridin-3-yl)methyl](methyl)oxido-.lamda.sup.4-sulphanylidene-cyanamide (known from WO 2007/149134), [1-(6-chloropyridin-3-yl)ethyl](methyl)oxido-.lamda.sup.4-sulphanylidene-cyanamide (known from WO 2007/149134) and its diastereomers (A) and (B) (likewise known from WO 2007/149134), [(6-trifluoromethylpyridin-3-yl)methyl](methyl)oxido-.lamda.sup.4-sulpha-nylidenecyanamide (known from WO 2007/095229), or [1-(6-trifluoromethylpyridin-3-yl)ethyl](methyl)oxido-.lamda.sup.4-sulph-anylidenecyanamide (known from WO 2007/149134) and its diastereomers (C) and (D), namely sulfoxaflor (likewise known from WO 2007/149134).

### EXAMPLES

Embodiments of this invention can be better understood by reference to the following examples. The foregoing and following description of embodiments of the invention and the various embodiments are not intended to limit the claims, but are rather illustrative thereof. Therefore, it will be understood that the claims are not limited to the specific details of these examples. It will be appreciated by those skilled in the art that other embodiments of the invention may be practiced without departing from the spirit and the scope of the disclosure, the scope of which is defined by the appended claims.

### Example 1

### Isolation of Bacillus DNA

Total DNA was isolated from Bacillus thuringiensis strains CGB316 and CGB323. Cultures of each strain were grown overnight in L-broth at 25° C. with 150 rpm of shaking in a rotary shaker. The 2 ml culture was centrifuged at 10K, and resuspended in 700 μl of: 8% sucrose, 100 mM tris pH 8.0, 10 mM EDTA, 50 mM NaCl and 2 mg/ml lysozyme. The resuspension was incubated for 30 minutes at 37° C. The solution was made to 50 g/ml proteinase K, and 20% SDS was added to 0.2% final concentration and incubated at 50° C. until the solution became very viscous. An equal volume of phenol/chloroform was added to the solution, which was vortexed and then centrifuged at 10K to separate the phases. The aqueous phase was then mixed with: 1 g/ml CsCl with 150 μg/ml ethidium bromide. The mixture was placed in an 33 ml Nalgene UltraLok tube, topped off with 1 g/ml CsCl, capped and centrifuged at 45,000 rpm for 16 hours in a Beckman Ti50 ultracentrifuge rotor. The DNA band was visualized with a UV light source and the band removed with a syringe using a 16 gauge needle. Ethidium bromide was removed by isoamyl alcohol extraction. DNA was precipitated with 2 volumes of 100% ethanol and centrifuged at 10K, then the DNA was washed with 70% ethanol, centrifuged, and the DNA pellet was dried at room temperature. The dry pellet was resuspended 2001 of 10 mM tris pH 8.0, 1 mM EDTA.

15 μg of DNA from CGB316 and CGB323 were each digested with 0.1 units Sau3A per 1 μg DNA at 37° C. 5 μg samples were removed at 3, 5 and 10 minutes after addition of restriction enzyme and samples 500 mM EDTA was added to make a final concentration of 10 mM EDTA and placed on ice. Samples were loaded on a 0.8% agarose gel utilizing a tris-borate-EDTA (TBE; Sambrook et al.) buffer and run overnight at 25 volts in an IBI model MPH gel electrophoresis system. Lambda DNA digested with HindIII was run as molecular weight markers. DNA fragments in the 6-9 kb range were cut out of the gel. These gel slices were placed in a Nanotrap electroelution trap and utilizing a ISCO Little Blue Tank with a buffer system and current described by the supplier, and thereby the DNA was electroeluted out of the agarose gel. DNA isolated from agarose was precipitated with addition of 1/10 volume 3M sodium acetate pH 4.8, 2.5 volumes 100% ethanol and centrifuged. The DNA was washed with 70% ethanol and centrifuged. The dry pellet was resuspended in 20 μl of 10 mM tris pH 8.0, 1 mM EDTA.

The resuspended DNA was ligated into pUC19. Ligations were done using 4 μl of the 6-9 kb DNA solution, 1 μl of 100 ng/μl of pUC19 digested with BamHI and treated with calf alkaline phosphatase, 1 μl 10× ligation buffer, 3 μl water, and 1 μl comprising 3 units T4 ligase. The ligation reaction was incubated at 15° C. overnight and then transformed into E. coli DH5 alpha competent cells by (a) mixing ligation mix with 200 μl cells and then placing on ice for 1-2 hours; (b) heating at 42° C. for 90 seconds; (c) mixing with 200 μl of SOC medium (Sambrook et al), and incubating at 37° C. for 45 minutes; and (d) plating the solution on L-agar plates with 100 μg/ml ampicillin (Sambrook et al). Plates were incubated overnight at 37° C.

Colony hybridizations were done as described in Sambrook, et al. Briefly, each plate had a 85 mm Nitroplus 2000 filter circle placed on it and then lifted off. After lifts the plates were incubated at 37° C. until colonies were visible again. The filters with the colonies on them were treated on Whatman paper saturated with 10% SDS, followed by 0.5 N NaOH-1.5 M NaCl, and then with 1.5 M NaCl-0.5 M tris pH 7.4, for 3 minutes each. Then the filters were saturated with 2×SSC, and DNA was fixed to the filters by UV crosslinking using a Stratalinker (Stratagene) at 0.2 mJoule. A total of 6 plates with 100-200 colonies/plate were done for each strain. Prehybridization and hybridization of the filter was carried out in a solution of 10×Denhardts solution, 150 μg/ml sheared salmon sperm DNA, 1% SDS, 50 mM sodium phosphate pH 7.0, 5 mM EDTA, 6×SSC, 0.05% sodium pyrophosphate. Prehybridization was at 65° C. for 4 hours and hybridization was at 65° C. for 18 hrs with 1 million cpm/ml of a ³²P-dCTP labeled probe in a volume of 50 ml. Radiolabeled DNA probes were prepared using a BRL random prime labeling system and unincorporated counts removed using Nick Columns (Pharmacia). Filters were probed with a PCR generated cryIB radiolabeled fragment that spans the region 461-1366 bp of the cry1B gene (SEQ ID NO: 7). Probes were boiled 5 minutes before addition to hybridization solution. Filters were washed twice in 50 ml of 2×SSC, 0.5% SDS at 65° C. for 20 minutes. Filters were exposed to Kodak X-Omat AR X-ray film with Dupont Cronex Lightning Plus intensifying screens at −80° C. Positives were identified and colonies picked and streaked on L-agar with 100 μg/ml ampicillin. Plasmid DNA was isolated using the alkaline miniprep method described in Molecular Cloning (Sambrook et al). The clone pCIB5618 containing 5618-cry1Ia (SEQ ID NO: 2) was digested with SacI/BbuI, and pCIB5621 containing 5621-cry1Ia (SEQ ID NO: 4) was digested with SmaI/BbuI. Each were then loaded on a 1% Seaplaque agarose gel using TBE buffer system and run overnight at 25 volts. The 7 kb DNA fragments for each clone was cut out.

Ligations were performed as described above using 5 μl of the melted (65° C.) agarose fragment, 4 μl of pHT3101 at 10 ng/μl (pHT3101 is a Bt/E. coli shuttle vector composed of pUC18, a Bt replicon and an erythromycin gene for selection in Bt [Lecadet et al 1992]); cut with either SacI/BbuI or SmaI/BbuI) in a total volume of 20 μl. E. coli was transformed as previously described. Plasmid DNA was then isolated, digested with appropriate restriction enzymes, and run on agarose-TBE gel to confirm the presence of the correct gene.

### Example 2

### Construction of Expression Vectors

Oligonucleotide primers were made spanning the promoter region of the cry1Ac promoter (SEQ ID NO: 8; Genbank accession number J01554). SEQ ID NO: 9 is a primer spanning nucleotides 1 to 20 of SEQ ID NO: 8, and SEQ ID NO: 10 is a primer spanning nucleotides 179 to 188 of SEQ ID NO: 8. Restriction sites were incorporated into the primers with an SfiI site at the 5′ end of the forward primer and an PmeI site at the 3′ end of the reverse primer. The 205 bp fragment was generated by PCR using the described primers. 100-250 ng of template DNA and each primer at 0.5 μM was added to a 0.5 ml GeneAmp reaction tube (Perkin Emler Cetus) containing 50 μl of PCR reaction mix as described in the GeneAmp Kit (Perkin Emler Cetus). AmpliTaq polymerase (2.5 units; Perkin Emler Cetus) was added to each tube. Amplification was accomplished with the GeneAmp PCR system 9600 (Perkin Elmer Cetus) using the Step-Cycle program set to denature at 94° C. for 45 seconds, anneal at 45° C. for 45 seconds, and extend at 72° C. for 1 min, followed by 4-seconds-per-cycle extension for a total of 35 cycles. Following amplification, the PCR reaction mix was analyzed by agarose gel electrophoresis as described by Sambrook et al. The PCR products were run on 1% Seaplaque (FMC Bioproducts) agarose-TBE gel. DNA fragments were cut out and electroeluted using ISCO Little Blue Tank (Lincoln, Nebr.) as described by the supplier. Samples were ethanol precipitated overnight at 4° C., washed with excess 70% ethanol and resuspended in 10 mM Tris pH 8.0, 1 mM EDTA.

The isolated DNA fragment was digested with the restriction enzymes SfiI and PmeI as described by the supplier. The digestion product was ligated into the plasmid pCIB5614 digested with the same restriction enzymes. The pCIB5614 containing the cry1Ac promoter was designated pCIB5634.

The cry1Ia like coding sequences were isolated from pCIB5618 and pCIB5621 by digesting with DraI which cuts 120 bp upstream of the ATG start codon and downstream of the TAG stop codon to yield a 3.8 kb fragment with blunt ends. This fragment was ligated into pCIB5634 cut with PmeI. These were transformed into DH5α E. coli cells as described in Sambrook, et al. Clones were plated on L agar plus 100 μg/ml ampicillin at 37° C. overnight. Positives were identified by the presence of crystal inclusions. The plasmid created by inserting the 5618-cry1Ia gene into pCIB5634 was designated pCIB7950 and the plasmid created by inserting the 5621-cry1Ia gene into pCIB5634 was designated pCIB7951.

### Example 3

### Transformation of Bacillus thuringiensis with Novel Genes

The host for the transformation was the acrystalliferous derivative CGB324. The method used was that of Schurter et al and is described below. 10 ml L-broth was inoculated with spores of HD73-1 02 and incubated overnight at 25° C. at 100 rpm on a rotary shaker. The overnight culture was diluted 50-fold into L-broth and incubated at 30° C. on a rotary shaker at 250 rpm until the culture reached an OD₅₅₀ of 0.2. Cells were harvested by centrifugation and resuspended in 1/40 volume ice-cold electroporation buffer (400 mM sucrose, 1 mM MgCl, 7 mM phosphate buffer pH 6.0, 20% glycerol). The centrifugation was repeated and the cells resuspended as described above. 400 μl of cells were added into a Genepulser with a 0.4 cm electrode gap, plasmid DNA was added and maintained at 4° C. for 10 minutes. The solution was electroporated with a capacitor of 25 Fad and 1300 volts by using the BioRad Genepulser transfection apparatus. After another 10 minute incubation at 4° C., the electroporated solution was diluted with 1.6 ml L-broth and incubated for 4 hours at 30° C. on a rotary shaker at 250 rpm. The culture was plated on T3 agar (3 g Tryptone, 2 g Tryptose, 1.5 g Yeast Extract, 0.05 g MgCl₂ 50 mM sodium phosphate pH 6.8) plus 25 μg/ml erythromycin and incubated at 30° C. for 24-36 hours to visualize colonies. Single colonies were streaked onto T3 plates plus erythromycin and grown to sporulation. Crystal producing colonies were identified microscopically.

### Example 4

### Bioassay of Cry1Ia-Like Genes Against Colorado Potato Beetle (CPB)

The CPB assays were performed in Gelman 50 mm petri dishes with a Gemen 47 mm filter paper circle to which 300 μl of distilled water has been added. The E. coli culture was diluted in 0.05% Triton X-100 at 1:1, 1:2, or 1:4 and 2.7 cm. Egg Plant leaf punches were dipped into the solution. These were allowed to dry in the petri dishes with the filter paper. Then five first instar CPB larvae were placed in each petri dish and the cover placed on top; 20 larvae were assayed per concentration. Petri dishes were placed in an incubator for 3 days at 72° F. with a 14:10 (hours) light:dark cycle. Then the number of live larvae in each cup was recorded.

Results are shown in Table 1. Unlike many known Cry1Ia toxins, the 5618-Cry1Ia and 5621-Cry1Ia toxins were not active against Leptinotarsa decemlineata (CPB), indicating that the amino acid differences are related to functional differences.

### Example 5

### Plutella xyostella Bioassay

Plutella bioassays were performed by incorporating aliquots of the overnight grown E. coli culture, comprising the cry1I-like endotoxin, with molten artificial P. xyostella diet (Biever and Boldt, Annals of Entomological Society of America, 1971; Shelton, et al J. Ent. Sci 26:17) and at appropriate high concentration. The 4 ml mixed toxic diet was poured into 1 oz. clear plastic cups (Bioserve product #9051). Subsequent dilutions were made by adding non-toxic diet to the previous concentration. Once the diet cooled, 5 neonate P. xyostella from a diet adapted lab colony were placed in each diet containing cup and then covered with a white paper lid (Bioserve product #9049). 20 larvae were assayed per concentration. Trays of the cups were placed in an incubator for 3 days at 72° C. with a 14:10 (hours) light:dark cycle. Then the number of live larvae in each cup was recorded.

TABLE 1

| Toxin | Px | On | Hz | Hv | Se | Sf | Ld |
|---|---|---|---|---|---|---|---|
| 5618-Cry1I | + | + | − | − | − | − | − |
| 5621-Cry1I | + | + | − | − | − | − | − |
| Known Cry1Ia | + | + | + | | + | + | + |
| Toxins* | | | | | | | |

*Information based on data reported in the literature.
Px = Plutella xyostella; On = Ostrinia nubilalis; Hz = Helicoverpa zea; Hv = Helicoverpa virescens; Se = Spodoptera exigua; Sf = Spodoptera frugiperda; Ld = Leptinotarsa decemlineata

### REFERENCES

Altschul et al., J. Mol. Biol. 215: 403-410 (1990)
Bevan et al. (1983) Nature 304:184-187
Biever and Boldt, Annals of Entomological Society of America, 1971
Binet et al., (1991) Plant Science 79: 87-94
Blochinger & Diggelmann, (1984) Mol Cell Biol 4: 2929-2931
Bourouis et al., 1983. EMBO J. 2(7): 1099-1104
Christensen et al., 1989. Plant Molec. Biol. 12: 619-632
Clonetech 1993/1994 catalog, page 210
Crickmore et al. (1998) Microbiol. Mol. Biol. Rev. 62:807-813
de Framond FEBS 290:103-106 (1991)
de Maagd et al. (2001) Trends Genetics 17:193-199
Feitelson (1993) The Bacillus Thuringiensis family tree. In Advanced Engineered Pesticides. Marcel Dekker, Inc., New York, N. Y.
Firek et al. Plant Molec. Biol. 22:129-142 (1993)
Goldschmidt-Clermont, M. (1991) Nucl. Acids Res. 19:4083-4089
Henikoff & Henikoff, Proc. Natl. Acad. Sci. USA 89: 10915 (1989)
Hudspeth & Grula, Plant Molec. Biol. 12:579-589 (1989)
Joshi NAR 15:6643-6653 (1987)
Karlin & Altschul, Proc. Nat'l. Acad. Sci. USA 90: 5873-5787 (1993)
Kostichka et al. (1996) J. Bacteriol. 178:2141-2144
Lecadet M M, Chaufaux J, Ribier J, Lereclus D. Appl Environ Microbiol. 1992 March; 58 (3):840-849
Logemann et al. Plant Cell 1:151-158 (1989)
McBride et al. (1994) Proc. Natl. Acad. Sci. USA 91, 7301-7305
McElroy et al. Mol. Gen. Genet. 231: 150-160 (1991)
Messing & Vierra., 1982. Gene 19: 259-268
Murray et al., Nucleic Acids Research 17:477-498 (1989)
Needleman & Wunsch, J. Mol. Biol. 48: 443 (1970)
Norris et al. 1993. Plant Molec. Biol. 21:895-906
Pearson & Lipman, Proc. Nat'l. Acad. Sci. USA 85: 2444 (1988)
Rohrmeier & Lehle, Plant Molec. Biol. 22:783-792 (1993)
Sambrook, Fritsch, and Maniatis. Molecular Cloning A Laboratory Manual, 2nd Ed., Cold Spring Harbor Laboratory Press (1989)
Schurter et al. Mol. Gen. Genet. (1989) 218:177-181
Shelton, et al J. Ent. Sci 26:17
Smith & Waterman, Adv. Appl. Math. 2: 482 (1981)
Spencer et al., 1990. Theor. Appl. Genet. 79: 625-631
Stanford et al. Mol. Gen. Genet. 215:200-208 (1989)
Staub, J. M., and Maliga, P. (1992) Plant Cell 4, 39-45
Staub, J. M., and Maliga, P. (1993) EMBO J. 12, 601-606
Svab, Z., Hajdukiewicz, P., and Maliga, P. (1990) Proc. Natl. Acad. Sci. USA 87, 8526-8530
Svab, Z., and Maliga, P. (1993) Proc. Natl. Acad. Sci. USA 90, 913-917
Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y.
Warner et al. Plant J. 3:191-201 (1993)
White et al., 1990. Nucl. Acids Res 18: 1062
Xu et al. Plant Molec. Biol. 22:573-588 (1993)
EP 0 332 104
EP 0 342 926
EP 0 359 472
EP 0 385 962
EP 0 452 269
U.S. Pat. No. 4,940,935
U.S. Pat. No. 5,188,642
U.S. Pat. No. 5,352,605
U.S. Pat. No. 5,451,513
U.S. Pat. No. 5,545,817
U.S. Pat. No. 5,545,818
U.S. Pat. No. 5,604,121
U.S. Pat. No. 5,614,395
U.S. Pat. No. 5,625,136
U.S. Pat. No. 5,767,378
U.S. Pat. No. 5,849,320
U.S. Pat. No. 5,849,870
U.S. Pat. No. 5,876,739
U.S. Pat. No. 5,877,012
U.S. Pat. No. 5,889,174
U.S. Pat. No. 5,994,629
U.S. Pat. No. 6,040,504
U.S. Pat. No. 6,107,279
U.S. Pat. No. 6,121,014
U.S. Pat. No. 6,137,033
U.S. Pat. No. 7,030,295
U.S. Pat. No. 7,230,167
WO 93/07278
WO 93/07278
WO 95/16783
WO 01/73087

1. A Cry1I toxin comprising 719 amino acids and having at least 99% identity with SEQ ID NO: 1, wherein the amino acid corresponding to position 140 is glutamic acid (E), the amino acid corresponding to position 184 is threonine (T), the amino acid corresponding to position 233 is aspartic acid (D), the amino acid corresponding to position 329 is isoleucine (I), the amino acid corresponding to position 377 is threonine (T), the amino acid corresponding to position 393 is phenylalanine (F) or leucine (L), the amino acid corresponding to position 549 is leucine (L), and the amino acid corresponding to position 712 is leucine (L) or glutamine (Q).

2. The Cry1I toxin of claim 1, wherein the Cry1I toxin is active against Lepidopteran insects and is not active against Coleopteran insects.

3. An isolated nucleic acid molecule comprising a nucleotide sequence that encodes the Cry1I toxin of claim 1.

4. The nucleic acid molecule of claim 3, wherein said nucleotide sequence is selected from the group consisting of SEQ ID NO: 2 and SEQ ID NO: 4.

5. The nucleic acid molecule of claim 3, wherein said nucleotide sequence has been codon optimized for expression in a plant.

6. The nucleic acid molecule of claim 5, wherein the optimized sequence comprises a sequence selected from the group consisting of SEQ ID NO: 5 and SEQ ID NO: 6.

7. A chimeric gene comprising a heterologous promoter sequence operatively linked to the nucleic acid molecule of claim 1.

8. The chimeric gene of claim 7, wherein said promoter is a plant-expressible promoter.

9. The chimeric gene of claim 8, wherein said plant-expressible promoter is selected from the group consisting of ubiquitin, cmp, corn TrpA, bacteriophage T3 gene 9 5′ UTR, corn sucrose synthetase 1, corn alcohol dehydrogenase 1, corn light harvesting complex, corn heat shock protein, pea small subunit RuBP carboxylase, Ti plasmid mannopine synthase, Ti plasmid nopaline synthase, petunia chalcone isomerase, bean glycine rich protein 1, Potato patatin, lectin, CaMV 35S, and the S-E9 small subunit RuBP carboxylase promoter.

10. A recombinant vector comprising the nucleic acid molecule of claim 1.

11. The vector of claim 10, further defined as a plasmid, cosmid, phagemid, artificial chromosome, phage or viral vector.

12. A transgenic non-human host cell comprising the chimeric gene of claim 7.

13. The transgenic non-human host cell of claim 12, wherein the transgenic non-human host cell is a transgenic plant cell.

14. The transgenic non-human host cell of claim 13, wherein the transgenic plant cell is selected from the group consisting of maize, wheat, rice, soybean, tobacco, and cotton.

15. A transgenic plant comprising the transgenic plant cell of claim 13.

16. The transgenic plant of claim 15, wherein the transgenic plant is selected from the group consisting of maize, wheat, rice, soybean, tobacco, and cotton.

17. A biological sample derived from the transgenic plant of claim 15, wherein said biological sample comprises the nucleic acid molecule or insecticidal protein.

18. The biological sample of claim 17, wherein said insecticidal protein protects said sample from insect infestation.

19. The biological sample of claim 17, wherein said sample is selected from the group consisting of flour, meal, oil, and starch, or a product derived therefrom.

20. A method of providing a farmer with a means of controlling a Lepidopteran insect pest, said method comprising supplying or selling to the farmer plant material, said plant material comprising a nucleic acid molecule capable of expressing the Cry1I toxin according to claim 1.

21. A method of producing the Cry1I toxin of claim 1, comprising the steps of: (a) transforming a non-human host cell with a recombinant nucleic acid molecule comprising a nucleotide sequence which codes for the Cry1I toxin; and (b) culturing the host cell of step (a) under conditions in which the host cell expresses the recombinant nucleic acid molecule, thereby producing the Cry1I toxin.

22. The method of claim 21, wherein the non-human host cell is a plant cell.

23. The method of claim 22, wherein the plant cell is a maize cell.

24. The method of claim 21, wherein the recombinant nucleic acid molecule is codon optimized for expression in a plant.

25. The method of claim 21, wherein the recombinant nucleic acid molecule comprises a sequence selected from the group consisting of SEQ ID NO: 2, SEQ ID NO: 4, SEQ ID NO: 5, and SEQ ID NO: 6.

26. The method of claim 25, wherein the recombinant nucleic acid molecule further comprises a promoter sequence operably linked to said nucleotide sequence to allow expression of the nucleotide sequence and production of the Cry1I toxin by the host cell.

27. The method of claim 21, wherein the transforming is performed by Agrobacterium-mediated transformation, electroporation, or microprojectile bombardment.

28. A method of reducing pest damage in a transgenic plant caused by Lepidopteran insects and Coleopteran insects, the method comprising planting a transgenic plant seed comprising a first transgene and a second transgene, wherein the first transgene causes expression of a Cry1I toxin and wherein the second transgene causes expression of a Cry3 toxin; thereby reducing damage caused by Lepidopteran insects and Coleopteran insects to a transgenic plant grown from the transgenic plant seed.

29. The method of claim 28, wherein the Cry1I toxin comprises 719 amino acids and having at least 99% identity with SEQ ID NO: 1, wherein the amino acid corresponding to position 140 is glutamic acid (E), the amino acid corresponding to position 184 is threonine (T), the amino acid corresponding to position 233 is aspartic acid (D), the amino acid corresponding to position 329 is isoleucine (I), the amino acid corresponding to position 377 is threonine (T), the amino acid corresponding to position 393 is phenylalanine (F) or leucine (L), the amino acid corresponding to position 549 is leucine (L), the amino acid corresponding to position 712 is leucine (L) or glutamine (Q).

30. The method of claim 28, wherein the first transgene comprises a nucleotide sequence selected from the group consisting of SEQ ID NO: 2, SEQ ID NO: 4, SEQ ID NO: 5 and SEQ ID NO: 6.

31. The method of claim 28, wherein the Cry3 toxin is a modified Cry3A toxin.

32. The method of claim 28, wherein the transgenic plant is selected from the group consisting of maize, wheat, rice, soybean, tobacco, and cotton.

33. The method of claim 32, wherein the transgenic plant is maize.

34. A transgenic seed from the transgenic plant of claim 15, wherein the transgenic seed comprises the nucleic acid molecule.

35. A method of controlling crop pests comprising providing the transgenic plant of claim 15 and applying to the plant an active ingredient selected from the group consisting of acetylcholine esterase (AChE) inhibitors, GABA-gated chloride channel antagonists, sodium-channel modulators/voltage-dependent sodium channel blockers, nicotinergic acetylcholine receptor agonists/antagonists, allosteric acetylcholine receptor modulators (agonists), chloride channel activators, active ingredients with unknown or nonspecific mechanisms of action, inhibitors of oxidative phosphorylation, ATP disruptors, oxidative phosphorylation decouplers which interrupt the H-proton gradient, microbial disruptors of the insect gut membrane, chitin biosynthesis inhibitors, moulting disruptors, ecdysone agonists/disruptors, octopaminergic agonists, site III electron transport inhibitors/site II electron transport inhibitors, ryanodin receptor effectors, fatty acid biosynthesis inhibitors, neuronal inhibitors with unknown mechanism of action and electron transport inhibitors.

36. The method of claim 35, wherein the active ingredient is selected from the group consisting of alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC and xylylcarb, acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-5-methyl, demeton-5-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, trichlorfon, vamidothion, imicyafos, camphechlor, chlordane, endosulfan, gamma-HCH, HCH, heptachlor, lindane, methoxychlor, acetoprole, ethiprole, fipronil, pyrafluprole, pyriprole, vaniliprole, acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrin (pyrethrum), eflusilanat; DDT, methoxychlor, acetamiprid, clothianidin, dinotefuran, imidacloprid, imidaclothiz, nitenpyram, nithiazine, thiamethoxam, AKD-1022, nicotine, bensultap, cartap, thiosultap-sodium, thiocylam, spinosad, spinetoram, abamectin, emamectin, emamectin benzoate, ivermectin, lepimectin, milbemectin, hydroprene, kinoprene, methoprene, epofenonane, triprene, fenoxycarb, pyriproxifen, diofenolan, methyl bromide, chloropicrin, sulphuryl fluoride, cryolite, pymetrozine, pyrifluquinazon, flonicamid, clofentezine, hexythiazox, etoxazole, diafenthiuron, azocyclotin, cyhexatin, fenbutatin oxide, propargite, tetradifon, chlorfenapyr, binapacyrl, dinobuton, dinocap, DNOC, Bacillus thuringiensis strains, bistrifluoron, chlorfluazuron, diflubenzuron, fluazuron, flucycloxuron, flufenoxuron, hexaflumuron, lufenuron, novaluron, noviflumuron, penfluoron, teflubenzuron, triflumuron, buprofezin, transfluthrin, acetamiprid, cyromazine, chromafenozide, halofenozide, methoxyfenozide, tebufenozide, fufenozide (JS118), azadirachtin, amitraz, hydramethylnon, acequinocyl, fluacrypyrim, cyflumetofen, cyenopyrafen, fenazaquin, fenpyroximate, pyrimidifen, pyridaben, tebufenpyrad, tolfenpyrad, rotenone, indoxacarb, metaflumizone, spirodiclofen, spiromesifen, tetramic acid, spirotetramat, bifenazate, flubendiamide, (R)-, (S)-3-chloro-N.sup.1-{2-methyl-4-[1,2,2,2-tetrafluoro-1-(trifluoromethyl)-ethyl]phenyl}-N.sup.2-(1-methyl-2-methylsulphonylethyl)phthalamide, chlorantraniliprole (Rynaxypyr), cyantraniliprole (Cyazypyr), amidoflumet, benclothiaz, benzoximate, bromopropylate, buprofezin, chinomethionat, chlordimeform, chlorobenzilate, clothiazoben, cycloprene, dicofol, dicyclanil, fenoxacrim, fentrifanil, flubenzimine, flufenerim, flutenzin, gossyplure, japonilure, metoxadiazone, petroleum, potassium oleate, pyridalyl, sulfluramid, tetrasul, triarathene and verbutin, thereby controlling the crop pests.

37. A method of controlling crop pests comprising providing the seed of claim 34 and applying to the seed an active ingredient selected from the group consisting of acetylcholine esterase (AChE) inhibitors, GABA-gated chloride channel antagonists, sodium-channel modulators/voltage-dependent sodium channel blockers, nicotinergic acetylcholine receptor agonists/antagonists, allosteric acetylcholine receptor modulators (agonists), chloride channel activators, active ingredients with unknown or nonspecific mechanisms of action, inhibitors of oxidative phosphorylation, ATP disruptors, oxidative phosphorylation decouplers which interrupt the H-proton gradient, microbial disruptors of the insect gut membrane, chitin biosynthesis inhibitors, moulting disruptors, ecdysone agonists/disruptors, octopaminergic agonists, site III electron transport inhibitors/site II electron transport inhibitors, ryanodin receptor effectors, fatty acid biosynthesis inhibitors, neuronal inhibitors with unknown mechanism of action and electron transport inhibitors.

38. The method of claim 37, wherein the active ingredient is selected from the group consisting of alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC and xylylcarb, acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-5-methyl, demeton-5-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, trichlorfon, vamidothion, imicyafos, camphechlor, chlordane, endosulfan, gamma-HCH, HCH, heptachlor, lindane, methoxychlor, acetoprole, ethiprole, fipronil, pyrafluprole, pyriprole, vaniliprole, acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrin (pyrethrum), eflusilanat; DDT, methoxychlor, acetamiprid, clothianidin, dinotefuran, imidacloprid, imidaclothiz, nitenpyram, nithiazine, thiamethoxam, AKD-1022, nicotine, bensultap, cartap, thiosultap-sodium, thiocylam, spinosad, spinetoram, abamectin, emamectin, emamectin benzoate, ivermectin, lepimectin, milbemectin, hydroprene, kinoprene, methoprene, epofenonane, triprene, fenoxycarb, pyriproxifen, diofenolan, methyl bromide, chloropicrin, sulphuryl fluoride, cryolite, pymetrozine, pyrifluquinazon, flonicamid, clofentezine, hexythiazox, etoxazole, diafenthiuron, azocyclotin, cyhexatin, fenbutatin oxide, propargite, tetradifon, chlorfenapyr, binapacyrl, dinobuton, dinocap, DNOC, Bacillus thuringiensis strains, bistrifluoron, chlorfluazuron, diflubenzuron, fluazuron, flucycloxuron, flufenoxuron, hexaflumuron, lufenuron, novaluron, noviflumuron, penfluoron, teflubenzuron, triflumuron, buprofezin, transfluthrin, acetamiprid, cyromazine, chromafenozide, halofenozide, methoxyfenozide, tebufenozide, fufenozide (JS118), azadirachtin, amitraz, hydramethylnon, acequinocyl, fluacrypyrim, cyflumetofen, cyenopyrafen, fenazaquin, fenpyroximate, pyrimidifen, pyridaben, tebufenpyrad, tolfenpyrad, rotenone, indoxacarb, metaflumizone, spirodiclofen, spiromesifen, tetramic acid, spirotetramat, bifenazate, flubendiamide, (R)-, (S)-3-chloro-N.sup.1-{2-methyl-4-[1,2,2,2-tetrafluoro-1-(trifluoromethyl)-ethyl]phenyl}-N.sup.2-(1-methyl-2-methylsulphonylethyl)phthalamide, chlorantraniliprole (Rynaxypyr), cyantraniliprole (Cyazypyr), amidoflumet, benclothiaz, benzoximate, bromopropylate, buprofezin, chinomethionat, chlordimeform, chlorobenzilate, clothiazoben, cycloprene, dicofol, dicyclanil, fenoxacrim, fentrifanil, flubenzimine, flufenerim, flutenzin, gossyplure, japonilure, metoxadiazone, petroleum, potassium oleate, pyridalyl, sulfluramid, tetrasul, triarathene and verbutin, thereby controlling the crop pests.


SEQUENCE LISTING (10 sequences)

SEQ ID NO: 1 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHESLESWVGNRNNTRARSVVKSQYIALELMFVQKLPSFAVSGEEVTLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAIVRNPHLLDFLEQVTIYSLLSRWSNTQYMNMWGGHKLEFRTIGGTLNTSTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRLNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKLLHIERNM

SEQ ID NO: 2 - DNA, 2160 bp, Bacillus thuringiensis
atgaaactaaagaatcaagataagcatcaaagtttttctagcaatgcgaaagtagataaaatctctacggattcactaaaaaatgaaacagatatagaattacaaaacattaatcatgaagattgtttgaaaatgtctgagtatgaaaatgtagagccgtttgttagtgcatcaacaattcaaacaggtattggtattgcgggtaaaatacttggtaccctaggcgttccttttgcaggacaagtagctagtctttatagttttatcttaggtgagctatggcctaaggggaaaaatcaatgggaaatctttatggaacatgtagaagagattattaatcaaaaaatatcaacttatgcaagaaataaagcacttacagacttgaaaggattaggagatgccttagctgtctaccatgaatcgctggaaagttgggttggaaatcgtaataacacaagggctaggagtgttgtcaagagccaatatatcgcattagaattgatgtttgttcagaaactaccttcttttgcagtgtctggagaggaggtaacattattaccgatatatgcccaagctgcaaatttacatttgttgctattacgtgatgcgtctatttttggaaaagagtggggattatcatcttcagaaatttcaacattttataaccgtcaagtcgaacgagcaggagattattccgaccattgtgtgaaatggtatagcacaggtctaaataacttgaggggtacaaatgccgaaagttgggtacgatataatcaattccgtagagacatgactttaatggtactagatttagtggcactatttccaagctatgatacacaaatgtatccaattaaaactacagcccaacttacaagagaagtatatacagacgcaattgggacagtacatcctcatccaagttttacaagtacgacttggtataataataatgcaccttcgttctctgccatagaggctgctattgttcgaaacccgcatctactcgattttctagaacaagttacaatttacagcttattaagtcgatggagtaacactcagtatatgaatatgtggggaggacataaactagaattccgaacaataggaggaacgttaaatacctcaacacaaggatctactaatacttctattaatcctgtaacattaccgttcacgtctcgagacgtctataggactgaatcattggcagggctaaatctatttttaactcaacctgttaatggagtacctagggttgattttcattggaaattcgtcacacatccgatcgcatctgataatttctattatccagggtatgctggaattgggacgcaattacaggattcagaaaatgaattaccacctgaagcaacaggacagccaaattatgaatcttatagtcatagattatctcatataggactcatttcagcatcacatgtgaaagcattggtatattcttggacgcatcgtagtgcagatcgtacaaatacaattgagccaaatagcattacacaaataccattagtaaaagcgttcaatctgtcttcaggtgccgctgtagtgagaggaccaggatttacaggtggggatatccttcgaagaacgaatactggtacatttggggatatacgattaaatattaatccaccatttgcacaaagatatcgcgtgaggattcgctatgcttctaccacagatttacaattccatacgtcaattaacggtaaagctattaatcaaggtaatttttcagcaactatgaatagaggagaggacttagactataaaacctttagaactgtaggctttaccactccatttagctttttagatgtacaaagtacattcacaataggtgcttggaacttctcttcaggtaacgaagtgtatatagatagaattgaatttgttccggtagaagtaacatatgaggcagaatatgattttgaaaaagcgcaagagaaggttactgcactgtttacatctacgaatccaagaggattaaaaacagatgtaaaggattatcatattgaccaggtatcaaatttagtagagtctctatcagatgaattctatcttgatgaaaagagagaattattcgagatagttaaatacgcgaagctactccatattgagcgtaacatgtag

SEQ ID NO: 3 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHESLESWVGNRNNTRARSVVKSQYIALELMFVQKLPSFAVSGEEVTLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAIVRNPHLLDFLEQVTIYSLLSRWSNTQYMNMWGGHKLEFRTIGGTLNTSTQGSTNTSINPVTLPLTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRLNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 4 - DNA, 2160 bp, Bacillus thuringiensis
atgaaactaaagaatcaagataagcatcaaagtttttctagcaatgcgaaagtagataaaatctctacggattcactaaaaaatgaaacagatatagaattacaaaacattaatcatgaagattgtttgaaaatgtctgagtatgaaaatgtagagccgtttgttagtgcatcaacaattcaaacaggtattggtattgcgggtaaaatacttggtaccctaggcgttccttttgcaggacaagtagctagtctttatagttttatcttaggtgagctatggcctaaggggaaaaatcaatgggaaatctttatggaacatgtagaagagattattaatcaaaaaatatcaacttatgcaagaaataaagcacttacagacttgaaaggattaggagatgccttagctgtctaccatgaatcgctggaaagttgggttggaaatcgtaataacacaagggctaggagtgttgtcaagagccaatatatcgcattagaattgatgtttgttcagaaactaccttcttttgcagtgtctggagaggaggtaacattattaccgatatatgcccaagctgcaaatttacatttgttgctattacgtgatgcgtctatttttggaaaagagtggggattatcatcttcagaaatttcaacattttataaccgtcaagtcgaacgagcaggagattattccgaccattgtgtgaaatggtatagcacaggtctaaataacttgaggggtacaaatgccgaaagttgggtacgatataatcaattccgtagagacatgactttaatggtactagatttagtggcactatttccaagctatgatacacaaatgtatccaattaaaactacagcccaacttacaagagaagtatatacagacgcaattgggacagtacatcctcatccaagttttacaagtacgacttggtataataataatgcaccttcgttctctgccatagaggctgctattgttcgaaacccgcatctactcgattttctagaacaagttacaatttacagcttattaagtcgatggagtaacactcagtatatgaatatgtggggaggacataaactagaattccgaacaataggaggaacgttaaatacctcaacacaaggatctactaatacttctattaatcctgtaacattaccgctcacgtctcgagacgtctataggactgaatcattggcagggctaaatctatttttaactcaacctgttaatggagtacctagggttgattttcattggaaattcgtcacacatccgatcgcatctgataatttctattatccagggtatgctggaattgggacgcaattacaggattcagaaaatgaattaccacctgaagcaacaggacagccaaattatgaatcttatagtcatagattatctcatataggactcatttcagcatcacatgtgaaagcattggtatattcttggacgcatcgtagtgcagatcgtacaaatacaattgagccaaatagcattacacaaataccattagtaaaagcgttcaatctgtcttcaggtgccgctgtagtgagaggaccaggatttacaggtggggatatccttcgaagaacgaatactggtacatttggggatatacgattaaatattaatccaccatttgcacaaagatatcgcgtgaggattcgctatgcttctaccacagatttacaattccatacgtcaattaacggtaaagctattaatcaaggtaatttttcagcaactatgaatagaggagaggacttagactataaaacctttagaactgtaggctttaccactccatttagctttttagatgtacaaagtacattcacaataggtgcttggaacttctcttcaggtaacgaagtgtatatagatagaattgaatttgttccggtagaagtaacatatgaggcagaatatgattttgaaaaagcgcaagagaaggttactgcactgtttacatctacgaatccaagaggattaaaaacagatgtaaaggattatcatattgaccaggtatcaaatttagtagagtctctatcagatgaattctatcttgatgaaaagagagaattattcgagatagttaaatacgcgaagcaactccatattgagcgtaacatgtag

SEQ ID NO: 5 - DNA, 2160 bp, Artificial Sequence
  codon optimized version of 5618-Cry1Ia
atgaagctgaagaaccaggacaagcaccagagcttcagcagcaacgccaaggtggacaagatcagcaccgacagcctgaagaacgagaccgacatcgagctgcagaacatcaaccacgaggactgcctgaagatgagcgagtacgagaacgtggagcccttcgtgagcgccagcaccatccagaccggcatcggcatcgccggcaagatcctgggcaccctgggcgtgcccttcgccggccaggtggccagcctgtacagcttcatcctgggcgagctgtggcccaagggcaagaaccagtgggagatcttcatggagcacgtggaggagatcatcaaccagaagatcagcacctacgcccgcaacaaggccctgaccgacctgaagggcctgggcgacgccctggccgtgtaccacgagagcctggagagctgggtgggcaaccgcaacaacacccgcgcccgcagcgtggtgaagagccagtacatcgccctggagctgatgttcgtgcagaagctgcccagcttcgccgtgagcggcgaggaggtgaccctgctgcccatctacgcccaggccgccaacctgcacctgctgctgctgcgcgacgccagcatcttcggcaaggagtggggcctgagcagcagcgagatcagcaccttctacaaccgccaggtggagcgcgccggcgactacagcgaccactgcgtgaagtggtacagcaccggcctgaacaacctgcgcggcaccaacgccgagagctgggtgcgctacaaccagttccgccgcgacatgaccctgatggtgctggacctggtggccctgttccccagctacgacacccagatgtaccccatcaagaccaccgcccagctgacccgcgaggtgtacaccgacgccatcggcaccgtgcacccccaccccagcttcaccagcaccacctggtacaacaacaacgcccccagcttcagcgccatcgaggccgccatcgtgcgcaacccccacctgctggacttcctggagcaggtgaccatctacagcctgctgagccgctggagcaacacccagtacatgaacatgtggggcggccacaagctggagttccgcaccatcggcggcaccctgaacaccagcacccagggcagcaccaacaccagcatcaaccccgtgaccctgcccttcaccagccgcgacgtgtaccgcaccgagagcctggccggcctgaacctgttcctgacccagcccgtgaacggcgtgccccgcgtggacttccactggaagttcgtgacccaccccatcgccagcgacaacttctactaccccggctacgccggcatcggcacccagctgcaggacagcgagaacgagctgccccccgaggccaccggccagcccaactacgagagctacagccaccgcctgagccacatcggcctgatcagcgccagccacgtgaaggccctggtgtacagctggacccaccgcagcgccgaccgcaccaacaccatcgagcccaacagcatcacccagatccccctggtgaaggccttcaacctgagcagcggcgccgccgtggtgcgcggccccggcttcaccggcggcgacatcctgcgccgcaccaacaccggcaccttcggcgacatccgcctgaacatcaacccccccttcgcccagcgctaccgcgtgcgcatccgctacgccagcaccaccgacctgcagttccacaccagcatcaacggcaaggccatcaaccagggcaacttcagcgccaccatgaaccgcggcgaggacctggactacaagaccttccgcaccgtgggcttcaccacccccttcagcttcctggacgtgcagagcaccttcaccatcggcgcctggaacttcagcagcggcaacgaggtgtacatcgaccgcatcgagttcgtgcccgtggaggtgacctacgaggccgagtacgacttcgagaaggcccaggagaaggtgaccgccctgttcaccagcaccaacccccgcggcctgaagaccgacgtgaaggactaccacatcgaccaggtgagcaacctggtggagagcctgagcgacgagttctacctggacgagaagcgcgagctgttcgagatcgtgaagtacgccaagctgctgcacatcgagcgcaacatgtaa

SEQ ID NO: 6 - DNA, 2160 bp, Artificial Sequence
  codon optimized version of 5618-Cry1Ia
atgaagctgaagaaccaggacaagcaccagagcttcagcagcaacgccaaggtggacaagatcagcaccgacagcctgaagaacgagaccgacatcgagctgcagaacatcaaccacgaggactgcctgaagatgagcgagtacgagaacgtggagcccttcgtgagcgccagcaccatccagaccggcatcggcatcgccggcaagatcctgggcaccctgggcgtgcccttcgccggccaggtggccagcctgtacagcttcatcctgggcgagctgtggcccaagggcaagaaccagtgggagatcttcatggagcacgtggaggagatcatcaaccagaagatcagcacctacgcccgcaacaaggccctgaccgacctgaagggcctgggcgacgccctggccgtgtaccacgagagcctggagagctgggtgggcaaccgcaacaacacccgcgcccgcagcgtggtgaagagccagtacatcgccctggagctgatgttcgtgcagaagctgcccagcttcgccgtgagcggcgaggaggtgaccctgctgcccatctacgcccaggccgccaacctgcacctgctgctgctgcgcgacgccagcatcttcggcaaggagtggggcctgagcagcagcgagatcagcaccttctacaaccgccaggtggagcgcgccggcgactacagcgaccactgcgtgaagtggtacagcaccggcctgaacaacctgcgcggcaccaacgccgagagctgggtgcgctacaaccagttccgccgcgacatgaccctgatggtgctggacctggtggccctgttccccagctacgacacccagatgtaccccatcaagaccaccgcccagctgacccgcgaggtgtacaccgacgccatcggcaccgtgcacccccaccccagcttcaccagcaccacctggtacaacaacaacgcccccagcttcagcgccatcgaggccgccatcgtgcgcaacccccacctgctggacttcctggagcaggtgaccatctacagcctgctgagccgctggagcaacacccagtacatgaacatgtggggcggccacaagctggagttccgcaccatcggcggcaccctgaacaccagcacccagggcagcaccaacaccagcatcaaccccgtgaccctgcccctgaccagccgcgacgtgtaccgcaccgagagcctggccggcctgaacctgttcctgacccagcccgtgaacggcgtgccccgcgtggacttccactggaagttcgtgacccaccccatcgccagcgacaacttctactaccccggctacgccggcatcggcacccagctgcaggacagcgagaacgagctgccccccgaggccaccggccagcccaactacgagagctacagccaccgcctgagccacatcggcctgatcagcgccagccacgtgaaggccctggtgtacagctggacccaccgcagcgccgaccgcaccaacaccatcgagcccaacagcatcacccagatccccctggtgaaggccttcaacctgagcagcggcgccgccgtggtgcgcggccccggcttcaccggcggcgacatcctgcgccgcaccaacaccggcaccttcggcgacatccgcctgaacatcaacccccccttcgcccagcgctaccgcgtgcgcatccgctacgccagcaccaccgacctgcagttccacaccagcatcaacggcaaggccatcaaccagggcaacttcagcgccaccatgaaccgcggcgaggacctggactacaagaccttccgcaccgtgggcttcaccacccccttcagcttcctggacgtgcagagcaccttcaccatcggcgcctggaacttcagcagcggcaacgaggtgtacatcgaccgcatcgagttcgtgcccgtggaggtgacctacgaggccgagtacgacttcgagaaggcccaggagaaggtgaccgccctgttcaccagcaccaacccccgcggcctgaagaccgacgtgaaggactaccacatcgaccaggtgagcaacctggtggagagcctgagcgacgagttctacctggacgagaagcgcgagctgttcgagatcgtgaagtacgccaagcagctgcacatcgagcgcaacatgtaa

SEQ ID NO: 7 - DNA, 3687 bp, Bacillus thuringiensis
ttgacttcaaataggaaaaatgagaatgaaattataaatgctgtatcgaatcattccgcacaaatggatctattaccagatgctcgtattgaggatagcttgtgtatagccgaggggaacaatatcgatccatttgttagcgcatcaacagtccaaacgggtattaacatagctggtagaatactaggcgtattgggcgtaccgtttgctggacaactagctagtttttatagttttcttgttggtgaattatggccccgcggcagagatcagtgggaaattttcctagaacatgtcgaacaacttataaatcaacaaataacagaaaatgctaggaatacggctcttgctcgattacaaggtttaggagattccttcagagcctatcaacagtcacttgaagattggctagaaaaccgtgatgatgcaagaacgagaagtgttctttatacccaatatatagctttagaacttgattttcttaatgcgatgccgcttttcgcaattagaaaccaagaagttccattattgatggtatatgctcaagctgcaaatttacacctattattattgagagatgcctctctttttggtagtgaatttgggcttacatcgcaggaaattcaacgctattatgagcgccaagtggaacgaacgagagattattccgactattgcgtagaatggtataatacaggtctaaatagcttgagagggacaaatgccgcaagttgggtacggtataatcaattccgtagagatctaacgttaggagtattagatctagtggcactattcccaagctatgacactcgcacttatccaataaatacgagtgctcagttaacaagagaagtttatacagacgcaattggagcaacaggggtaaatatggcaagtatgaattggtataataataatgcaccttcgttctctgccatagaggctgcggctatccgaagcccgcatctacttgattttctagaacaacttacaatttttagcgcttcatcacgatggagtaatactaggcatatgacttattggcgggggcacacgattcaatctcggccaataggaggcggattaaatacctcaacgcatggggctaccaatacttctattaatcctgtaacattacggttcgcatctcgagacgtttataggactgaatcatatgcaggagtgcttctatggggaatttaccttgaacctattcatggtgtccctactgttaggtttaattttacgaaccctcagaatatttctgatagaggtaccgctaactatagtcaaccttatgagtcacctgggcttcaattaaaagattcagaaactgaattaccaccagaaacaacagaacgaccaaattatgaatcttacagtcacaggttatctcatataggtataattttacaatccagggtgaatgtaccggtatattcttggacgcatcgtagtgcagatcgtacgaatacgattggaccaaatagaatcacccaaatcccaatggtaaaagcatccgaacttcctcaaggtaccactgttgttagaggaccaggatttactggtggggatattcttcgaagaacgaatactggtggatttggaccgataagagtaactgttaacggaccattaacacaaagatatcgtataggattccgctatgcttcaactgtagattttgatttctttgtatcacgtggaggtactactgtaaataattttagattcctacgtacaatgaacagtggagacgaactaaaatacggaaattttgtgagacgtgcttttactacaccttttacttttacacaaattcaagatataattcgaacgtctattcaaggccttagtggaaatggggaagtgtatatagataaaattgaaattattccagttactgcaaccttcgaagcagaatatgatttagaaagagcgcaagaggcggtgaatgctctgtttactaatacgaatccaagaagattgaaaacagatgtgacagattatcatattgatcaagtatccaatttagtggcgtgtttatcggatgaattctgcttggatgaaaagagagaattacttgagaaagtgaaatatgcgaaacgactcagtgatgaaagaaacttactccaagatccaaacttcacatccatcaataagcaaccagacttcatatctactaatgagcaatcgaatttcacatctatccatgaacaatctgaacatggatggtggggaagtgagaacattaccatccaggaaggaaatgacgtatttaaagagaattacgtcacactaccgggtacttttaatgagtgttatccgacgtatttatatcaaaaaataggggagtcggaattaaaagcttatactcgctaccaattaagaggttatattgaagatagtcaagatttagagatatatttgattcgttataatgcgaaacatgaaacattggatgttccaggtaccgagtccctatggccgctttcagttgaaagcccaatcggaaggtgcggagaaccgaatcgatgcgcaccacattttgaatggaatcctgatctagattgttcctgcagagatggagaaaaatgtgcgcatcattcccatcatttctctttggatattgatgttggatgcacagacttgcatgagaatctaggcgtgtgggtggtattcaagattaagacgcaggaaggtcatgcaagactagggaatctggaatttattgaagagaaaccattattaggagaagcactgtctcgtgtgaagagggcagagaaaaaatggagagacaaacgtgaaaaactacaattggaaacaaaacgagtatatacagaggcaaaagaagctgtggatgctttattcgtagattctcaatatgatagattacaagcggatacaaacatcggcatgattcatgcggcagataaacttgttcatcgaattcgagaggcgtatctttcagaattacctgttatcccaggtgtaaatgcggaaatttttgaagaattagaaggtcacattatcactgcaatctccttatacgatgcgagaaatgtcgttaaaaatggtgattttaataatggattaacatgttggaatgtaaaagggcatgtagatgtacaacagagccatcatcgttctgaccttgttatcccagaatgggaagcagaagtgtcacaagcagttcgcgtctgtccggggtgtggctatatccttcgtgtcacagcgtacaaagagggatatggagagggctgcgtaacgatccatgaaatcgagaacaatacagacgaactaaaatttaaaaaccgtgaagaagaggaagtgtatccaacggatacaggaacgtgtaatgattatactgcacaccaaggtacagctggatgcgcagatgcatgtaattcccgtaatgctggatatgaggatgcatatgaagttgatactacagcatctgttaattacaaaccgacttatgaagaagaaacgtatacagatgtaagaagagataatcattgtgaatatgacagagggtatgtcaattatccaccagtaccagctggttatgtgacaaaagaattagaatacttcccagaaacagatacagtatggattgagattggagaaacggaaggaaagtttattgtagatagcgtggaattactcctcatggaagaatag

SEQ ID NO: 8 - DNA, 188 bp, Bacillus thuringiensis
tggcctatatatggcctgggcgagaagtaagtagattgttaacaccctgggtcaaaaattgatatttagtaaaattagttgcactttgtgcattttttcataagatgagtcatatgttttaaattgtagtaatgaaaaacagtattatatcataatcaattggtatcttaataaaagagatggaggtt

SEQ ID NO: 9 - DNA, 20 bp, Artificial Sequence
  This is a primer useful in the invention.
tggcctatatatggcctggg

SEQ ID NO: 10 - DNA, 10 bp, Artificial Sequence
  This is a primer useful in the invention.
gatggaggtt